(19) United States
(12) Patent Application Publication
(10) Pub. No.: US 2019/0237172 A1
(43) Pub. Date: Aug. 1, 2019

(54) HEALTHCARE OPTIMIZATION SYSTEMS AND METHODS TO PREDICT AND OPTIMIZE A PATIENT AND CARE TEAM JOURNEY AROUND MULTI-FACTOR OUTCOMES

(71) Applicant: ArmadaGlobal, Hunt Valley, MD (US)

(72) Inventors: Bharath Sudharsan, Abingdon, MD (US); Suzanne Sysko Clough, Wilmington, DE (US)

(21) Appl. No.: 16/380,032

(22) Filed: Apr. 10, 2019

Related U.S. Application Data
(60) Provisional application No. 62/655,321, filed on Apr. 10, 2018.

(51) Int. Cl. G16H 10/60; G16H 50/20; G16H 50/50; G16H 50/70

(52) U.S. Cl. CPC G16H 10/60; G16H 50/20; G16H 40/20; G16H 50/70; G16H 50/50

(57) ABSTRACT

Systems and methods for patient healthcare plan optimization. An optimization system includes a user interface, an optimizer and an influencer system. The user interface receives data including multi-factor determinants of a patient and a plurality of treatment personnel. The multi-factor determinants include at least one of clinical, behavioral, psychosocial, organizational and economic characteristics. The optimizer generates an electronic patient journey plan for the patient, by identifying one or more personnel among the treatment personnel to form a care team, roles for each selected personnel of the care team and actions for each of the care team and the patient, based on optimization of the multi-factor determinants of the patient and the treatment personnel according to one or more optimization algorithms. The influencer system determines software influencer instructions to be performed by designated influencers based on the electronic patient journey plan.

### TECHNICAL FIELD

The present disclosure relates generally to patient healthcare management techniques and, in particular, to systems and methods of predicting and optimizing a patient's healthcare journey and a care team's journey around multi-factor (e.g., clinical, behavioral, psychosocial, organizational and economic) outcomes.

### BACKGROUND

Patient healthcare management systems are known. Conventional solutions focus on using clinical workflows and behavioral change interventions to optimize clinical and behavioral outcomes of the patient's healthcare. These solutions, however don't focus on social, behavioral and psychosocial attributes of both the patient and the care team, including the physician. Instead, conventional solutions focus solely on the patient. Current solutions also do not predict a patient's healthcare progress and its effect on outcomes. Hence, most interventions, in conventional techniques, occur after the patient is referred to a physician, and in many cases the ability to influence the patient's healthcare outcomes is limited. Also, conventional management systems do not adapt and course-correct the interventions based on the patient's healthcare progress over time. Yet further, conventional management systems do not consider optimizing outcome around quality of life outcomes for the care team and the physician (e.g., in addition to any quality of life considerations of the patient). Moreover, management systems focus on a standard list of clinical and social determinants. Conventional systems do not consider various irrational personality-based and family-specific determinants (e.g., for the patient, physician and care team) that could influence outcomes of the patient's healthcare progress.

### SUMMARY

Aspects of the present disclosure relate to systems, methods and non-transitory computer readable mediums for creating an optimized electronic patient healthcare journey plan. An optimization system includes a user interface, an optimizer and an influencer system. The user interface is configured to receive data comprising multi-factor determinants of a patient and a plurality of treatment personnel. The multi-factor determinants includes at least one of clinical, behavioral, psychosocial, organizational and economic characteristics. The optimizer is configured to generate an electronic patient journey play for the patient, by identifying one or more personnel among the plurality of treatment personnel to form a care team, one or more roles for each selected personnel of the care team and one or more actions for each of the care team and the patient, based on optimization of the multi-factor determinants of the patient and the plurality of treatment personnel according to one or more optimization algorithms. The influencer system is configured to determine one or more software influencer instructions to be performed by one or more designated influencers based on the electronic patient journey plan.

### BRIEF DESCRIPTION OF DRAWINGS

FIG. 1 is a functional block diagram of an example journey optimization system, according to an aspect of the present disclosure.

FIG. 2 is a functional block diagram of example inputs to a user interface associated with the system shown in FIG. 1, according to an aspect of the present disclosure.

FIG. 3 is a functional block diagram of an example data warehouse associated with the system shown in FIG. 1, according to an aspect of the present disclosure.

FIG. 4 is a functional block diagram of an example role-based optimizer associated with the system shown in FIG. 1, according to an aspect of the present disclosure.

FIG. 5 is a functional block diagram of an example influencer system associated with the system shown in FIG. 1, according to an aspect of the present disclosure.

FIG. 6 is a flow chart diagram of an example method for journey optimization for care stakeholders, associated with the system shown in FIG. 1, according to an aspect of the present disclosure.

FIG. 7 is a functional block diagram of an example computer system, according to an aspect of the present disclosure.

### DETAILED DESCRIPTION

Aspects of the present disclosure relate to systems and methods for creating an optimized electronic patient healthcare journey plan that takes into consideration multiple factors, including factors relating to the patient, physician(s) and care team for the patient. A journey optimization system of the present disclosure may predict and optimize both the patient's journey and the care team's journey around clinical, behavioral, psychosocial, organizational and economic outcomes. Such optimization may include recommending the most effective care team for a given patient.

As defined herein, a journey may include one or more steps involved for a patient to navigate through a healthcare process, including identifying an appropriate institution, physician and/or care team. The journey may also include all experiences involved in each step and its effect on each person involved at the respective step. These experiences may include those of the patient, the physician and the care team. Experience may include, without being limited to, interactions, attitudes, beliefs, perceptions, physiological, behavioral and clinical changes. The experiences may be communicated or shared via one or more channels for the system to optimize the overall journey for the patient, as well as for scoring each person involved, and for optimized future interactions for other patients as well.

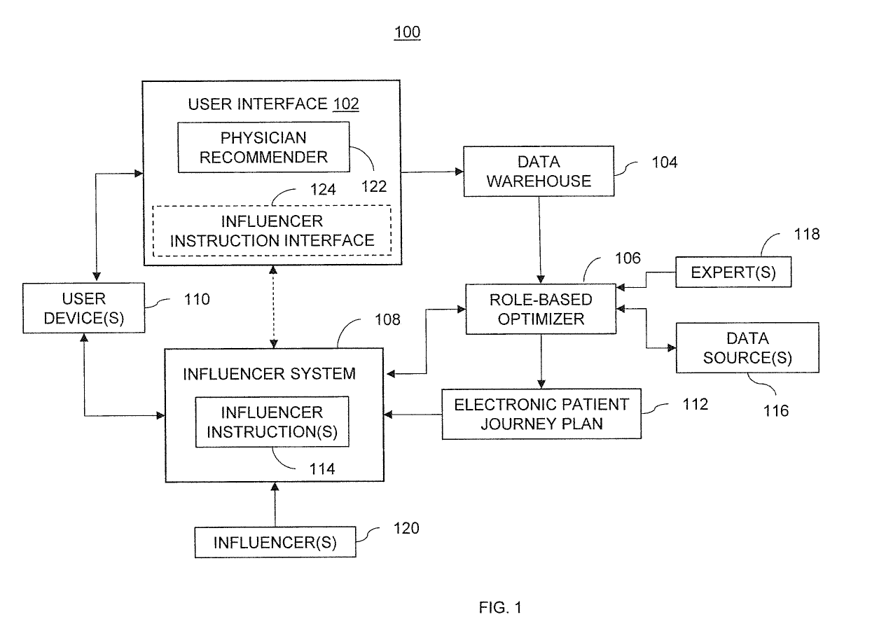

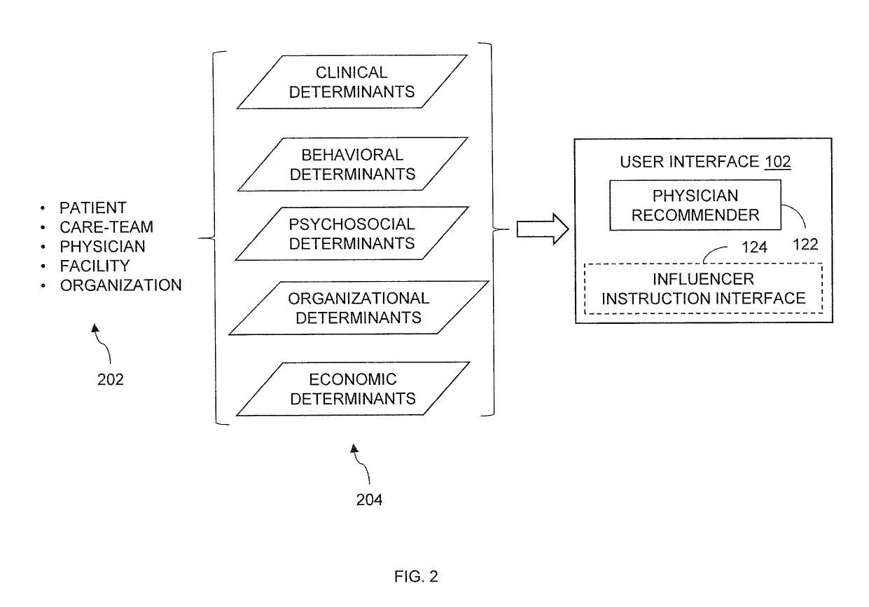

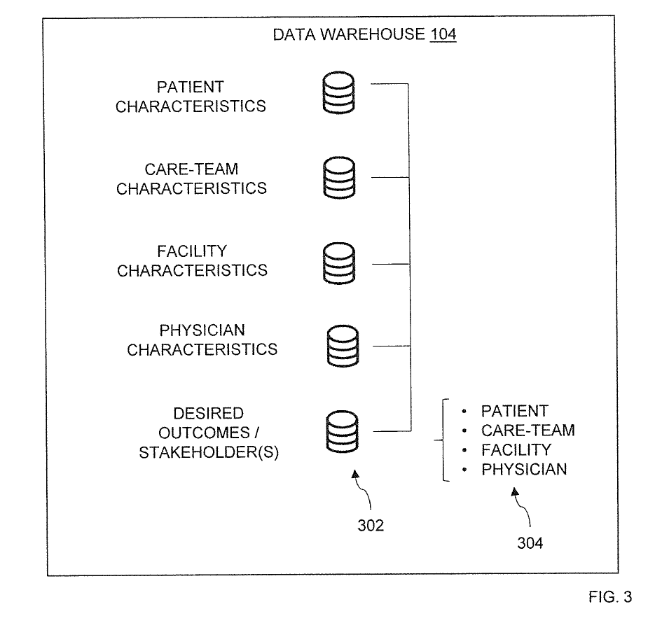

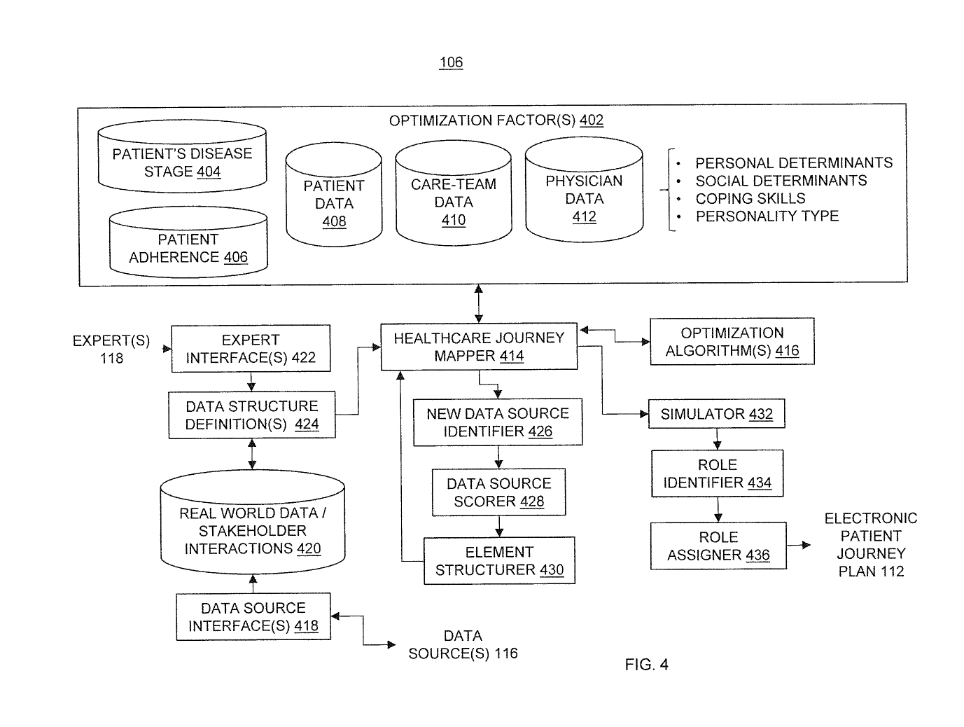

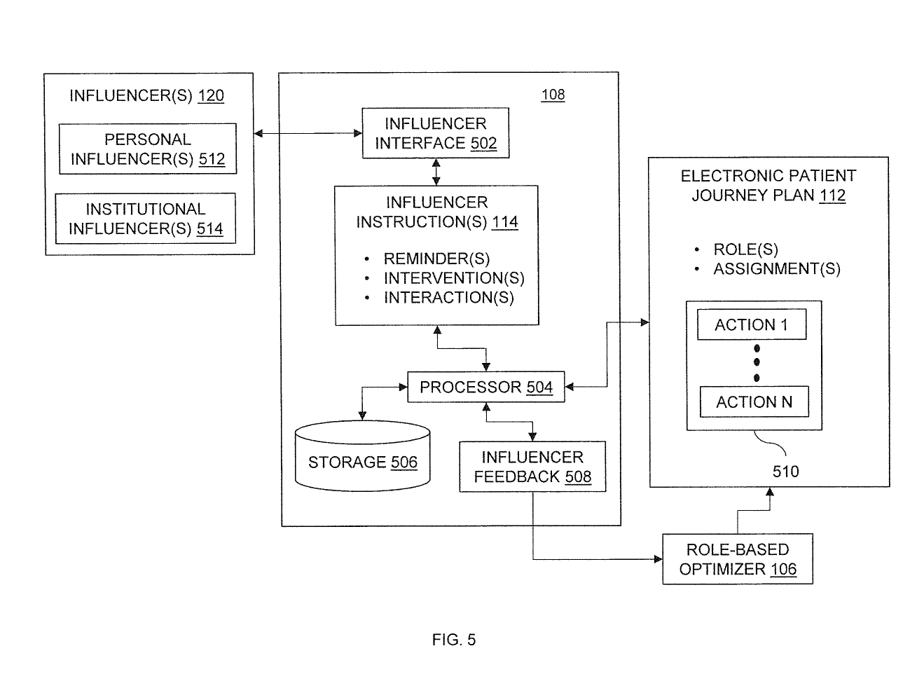

Referring to FIGS. 1-5, journey optimization system 100 (also referred to herein as system 100) is described, according to aspects of the present disclosure. In particular, FIG. 1 is a functional block diagram illustrating example system 100; FIG. 2 is a functional block diagram of example inputs 204 to user interface 102 of system 100; FIG. 3 is a functional block diagram of example data warehouse 104 of system 100; FIG. 4 is a functional block diagram of example role-based optimizer 106 of system 100; and FIG. 5 is a functional block diagram of example influencer system 108 of system 100.

As shown in FIG. 1, system 100 may include user interface 102, data warehouse 104, role-based optimizer 106 and influencer system 108. System 100 may communicate with one or more user device(s) 110, for example, via user interface 102, via role-based optimizer 106 and/or via influencer system 108. For example, user device(s) 110 may communicate with one or more components of system 100 (e.g., data warehouse 104, role-based optimizer 106 and/or influencer system 108) via user interface 102. As another example, user device(s) 110 may directly communicate with one or more components of system 100 (e.g., data warehouse 104, role-based optimizer 106 and/or influencer system 108).

In some examples, system 100 may communicate with and obtain data from one or more data source(s) 116. In some examples, role-based optimizer may be configured to interact with one or more experts 118 (e.g., data scientist(s), subject matter expert(s), etc., described further below). In some examples, influencer system 108 may be configure to interact with one or more influencer(s) 120 (described further below).

Each of user interface 102, data warehouse 104, role-based optimizer 106, influencer system 108 and user device(s) 110 may comprise one or more computing devices, including a non-transitory memory storing computer-readable instructions executable by a processing device to perform the functions described herein. It should be understood that journey optimization system 100 refers to a computing system having sufficient processing and memory capabilities to perform the specialized functions described herein.

Although not shown, system 100 may include a controller specially configured to control operation of user interface 102, data warehouse 104, role-based optimizer 106 and/or influencer system 108. The controller may include, for example, a processor, a microcontroller, a circuit, software and/or other hardware component(s).

In some examples, components of journey optimization system 100 (e.g., user interface 102, data warehouse 104, role-based optimizer 106 and influencer system 108) may be embodied on a single computing device. In other examples, journey optimization system 100 may refer to two or more computing devices distributed over several physical locations, connected by one or more wired and/or wireless links.

User interface 102, data warehouse 104, role-based optimizer 106, influencer system 108, user device(s) 110 and data source(s) 116 may be communicatively coupled via one or more networks (not shown). The one or more networks may include, for example, a private network (e.g., a local area network (LAN), a wide area network (WAN), intranet, etc.) and/or a public network (e.g., the Internet).

User device(s) 110 may comprise a desktop computer, a laptop, a smartphone, tablet, or any other user device known in the art. A user may interact with user device(s) 110, for example, via a graphical user interface (e.g., user interface 102) displayed on any type of display device including a computer monitor, a smart-phone screen, tablet, a laptop screen or any other device providing information to a user. User device(s) 110 may include any suitable user interface, user input component(s), output component(s), and communication component(s) for creation, transmission and receipt of electronic information and data related to data entry, data manipulation and data/information output (such as electronic patient journey plan 112 and influencer instruction(s) 114). Users of system 100 may include, without being limited to, patients, care teams, physicians, data scientists, subject matter experts, facility personnel and/or organizational personnel.

User interface 102 may include physician recommender 122 for receiving data and/or information. As shown in FIG. 2, user interface 102 may be configured to receive data and/or information (collectively referred to as data/information 204) from various users 202 (e.g., patients, care teams, physicians, facility personnel, organizational personnel, data scientists, subject matter experts) for entry and/or manipulation by various components of system 100. Non-limiting examples of such data/information 204 may include clinical determinants, behavior determinants, psychosocial determinants, organizational determinants and economic determinants. As shown in FIGS. 1 and 2, user interface 102 may provide data/information 204 to data warehouse 104.

Referring to FIGS. 1 and 2, user interface 102 may include physician recommender 122 for obtaining data/information 204. In some examples, physician recommender 122 may be configured to generate a specialized graphical user interface (GUI) for the presentation, input, manipulation and/or selection of data/information 204 in one or more windows of a display screen (not shown) of user interface 102. In some examples, physician recommender 122 may include a software application having specially programmed instructions configured to render the GUI.

In some examples, user interface 102 may also be configured to display results determined by system 100, including, without being limited to, electronic patient journey plan 112 and influencer instruction(s) 114. In some examples, user interface 102 may include optional influencer instruction interface 124 for communicating results determined by system 100. In some examples, the results may be rendered in physician recommender 122. In some examples, optional influencer instruction interface 124 may include a specialized software application for rendering a specific GUI for the presentation of the results, in one or more windows of a display screen.

Referring to FIG. 3, data warehouse 104 may include one or more databases 302 for storing various data/information from users of system 100. Data warehouse 104, in general, may store all metadata for available stakeholders 304 (e.g., patients, care team, facility, physicians). For example, data warehouse 104 may store patient characteristics, care team characteristics, facility characteristics, physician characteristics and desired outcomes/stakeholder information for each stakeholder 304. As shown in FIG. 1, data/information stored in data warehouse 104 may be provided to role-based optimizer 106.

Referring to FIG. 4, role-based optimizer 106 may include one or more optimization factors 402, healthcare journey mapper 414 (also referred to herein as mapper 414), one or more optimization algorithms 416, at least one data source interface 418, storage 420, at least one expert interface 422, one or more data structure definitions 424, new source identifier 426, data source scorer 428, element structurer 430, simulator 432, role identifier 434 and role assigner 436. In some examples, optimization algorithm(s) 416 may perform machine learning, artificial intelligence (AI) and/or statistical processing techniques. Role-based optimizer 106, via healthcare journey mapper 414, may perform a variety of tasks including organizing available data, identifying and capturing new applicable data as well as running simulations to create electronic patient journey plan 112. Electronic patient journey plan 112 may contain desirable roles, assignments for each role and one or more actions 510 (see FIG. 5, where n is any positive integer greater than or equal to one).

In some examples, healthcare journey mapper 414 may include a controller specially configured to control operation of components 402 and 416-436 of role-based optimizer 106. In some examples, healthcare journey mapper 414 may include, for example, a processor, a microcontroller, a circuit, software and/or other hardware component(s).

Data source interface(s) 418 may be configured to communicate with data source(s) 116, in order to obtain real-world data on stakeholder interactions from among data source(s) 116. Data source(s) 116 may include any suitable source of data for obtaining stakeholder interactions (e.g., interactions between patients and various physicians). For example, data source(s) 116 may include, without being limited to, electronic medical data systems, behavioral data systems, invasive or non-invasive wearable and/or monitoring devices, electronic databases associated with one or more of an insurance organization, a hospital, a physician medical practice, an outpatient clinic and an urgent care facility, social media, news sources, etc. The obtained real-world data may be stored in storage 420. Storage 420 may include any suitable non-transitory computer readable storage medium for receiving, storing and retrieving electronic data. Storage 420 may include, without being limited to, at least one of a database, a read-only memory (ROM), a random access memory (RAM), a flash memory, a dynamic RAM (DRAM) and a static RAM (SRAM).

Expert interface(s) 422 may be configured for interaction with expert(s) 118 (for example, data scientist(s), subject matter experts, etc.). Expert interface(s) 422 may be configured to provide at least a portion of the real world data stored in storage 420 for review and/or analysis by expert(s) 118, and to receive definitions of data elements and data source(s) 116 associated with the analyzed portion of real world data, to form data structure definition(s) 424. In some examples, data structure definition(s) 424 may be stored in storage 420. In some examples, expert interface(s) 422 may be configured to provide a user interface, such as a GUI for interaction with expert(s) 118. In some examples, expert interface 422 may be configured to present and/or allow manipulation of different information depending on the type of expert interacting with role-based optimizer 106.

Data structure definition(s) 424 may indicate, for example, one or more entities, attributes, relationships, etc. associated with the data elements. Based on data structure definition(s) 424, the data elements may be organized, via element structurer 430, according to one or more data models, as structured data elements. In general, a structured data element may include one or more embedded data types (such as one or more child elements) which may be based on data structure definition(s) 424. In general, the creation and use of structured data provides advantages with respect to organizing, storing, querying and analyzing the data.

Role-based optimizer 106 may comprise an analytical sub-system (i.e., healthcare journey mapper 414) capable of optimizing various determinants around clinical, behavioral, psychosocial, organizational and economic outcomes, both at an institutional and role-specific level. One example of such an optimization may be performed with respect to a case mix. For example, a desired and defined distribution of patient count around various diagnoses and procedures that a physician is capable of and interested in treating. Another example optimization may be performed with respect to various qualitative (e.g., quality of life, reduced burden of stress) and quantitative (e.g., net earnings, surgery volume) outcomes. Another example optimization may be performed with respect to patient recovery, satisfaction and holistic wellness.

As part of the optimization, healthcare journey mapper 414 may obtain one or more optimization factors 402. Optimization factors 402 may be obtained from data warehouse 104 and stored among storage(s) 404-412. Factors 402 that may contribute to an optimization may be based on, without being limited to, each stakeholder's (e.g., patient, care team and physician) personal and social determinants, coping skills, stage of disease and/or diagnosis, adherence, personality type etc. For example, optimization factors 402 may be selected from among patient's disease stage storage 404, patient adherence storage 406, patient data storage 408, care-team data storage 410 and physician data storage 412. Each of patient data, care-team data and physician data may include one or more of personal determinants, social determinants, coping skills and personality type information. In some examples, storage 404-412 may represent one storage device (e.g., one database). In some examples, storage 404-412 may represent more than one storage device (e.g., at least two databases).

Role-based optimizer 106 may simulate one or more possible interactions between each person (i.e., stakeholder) and/or with other entities at least based in part on optimization factor(s) 402. Role-based optimizer 106 may also perform a validation using real-world data collected at personal, social and organizational level, over a period of time, via direct and/or indirect methods (which data may be stored in storage 420). In some example, the data collection may involve use of data source(s) 116 such as, without being limited to, an electronic medical data system, a behavioral data system and/or an invasive or non-invasive wearable and/or monitoring device.

Role-based optimizer 106 may learn via algorithm(s) 416, which may include machine learning algorithms, AI algorithms and other suitable algorithms, and update weights and/or other related parameters for one or more models and/or rules, incrementally or in batches of interactions and respective data. In some examples, the updates may occur in real-time or near real-time in a secure software platform based on one or more technologies (for example blockchain).

The data elements and data sources used by role-based optimizer 106 for optimization and learning may be defined (e.g., as data structure definition(s) 424) by one or more expert(s) 118 (e.g., data scientists and/or clinical and behavioral subject matter experts). Based on the records of interactions created between system 100 and expert(s) 118 (via expert interface 422), healthcare journey mapper 414 may identify common themes, topics and semantics between data elements and data sources. Based on this intelligence, and by using one or more web discovery and web scraping technologies, role-based optimizer 106 may automatically identify the most relevant data sources and data elements (e.g., real world data on stakeholder interactions obtained from data source(s) 116). In some examples, mapper 414 may also retrieve, filter and store the data (e.g., in storage 420), by using metadata, in the context of a journey optimization and care team matching.

Role-based optimizer 106 may include new data source identifier 426 to identify new source(s) of data (e.g., from among data sources 116) that may be relevant, such as by using web discovery and/or web scraping technologies. Data source scorer 428 may be configured to score the new data source(s) and element structurer 430 may be configured to structure the new data source(s), such as via optimization algorithm(s) 416. For example, data source scorer 428, via mapper 414, may score new data sources in terms of novelty and incremental utility to one or more outcomes, in order to intelligently prioritize retrieval and storage efforts.

In some examples, mapper 414 may track and predict a server runtime, data storage capacity and pre-processing computing efforts (based on examples of such workflows in the past) of one or more components of system 100, and compute cost-benefit ratios (e.g., benefit may be calculated based on pre-defined business rules and updated over time based on machine learning), in order to prioritize data retrieval and storage tasks for various data sources 116 across the internet (or other network(s)) and/or across various identified organizations.

Simulator 432 may be configured to execute one or more simulation processes, according to structured data elements identified by mapper 414 as being applicable for a particular desired outcome. Simulation process(s) of simulator 432 may be based on one or more predetermined models and or predetermined rules. The simulation process(s) performed by simulator 432 may simulate possible interaction(s) between each stakeholder, based on the applicable and structured data elements (e.g., stored in storage 420), in order to optimize particular determinants (e.g., among optimization factor(s) 402) based on a desired outcome. Role identifier 434 may be configured to identify desired roles and/or desired stakeholders (e.g., based on one more predefined thresholds) based on an optimized outcome of simulator 432. Role assigner 436 may be configured to identify assignments and/or tasks for each identified role, for each identified stakeholder.

Role-based optimizer 106, via mapper 414, simulator 432, role identifier 436 and role assigner 436, may be configured based on each specific role or person involved during the patient journey plan, and from each person's perspective. Role-based optimizer 106 may be configured to optimize outcomes based on respective data elements needed for informing about various key clinical, behavioral and psychological insights and decisions that could influence overall outcomes. Role-based optimizer 106, based on the optimized outcomes, may generate electronic patient journey plan 112. In some examples, role assigner 436 (or a combination of simulator 432, role identifier 434 and role assigner 436) may combine and package the identified roles, assignments and one or more actions (e.g., tasks) 510 (see FIG. 5), across all desired stakeholders to form electronic patient journey plan 112, and may send electronic patient journey plan 112 to influencer system 108 (FIG. 1).

Referring to FIG. 5, influencer system 108 may include, influencer interface 502, processor 504 and storage 506. In some examples, processor 504 may be configured to control operation of one or more of influencer interface 502 and storage 506. Processor 504 may also be configured to communicate with role-based optimizer 106 (e.g., via an interface, not shown).

Influencer interface 502 may be configured to present data/information to influencer(s) 120 and to receive data/information from influencer(s) 120 for generating, updating and/or modifying influencer instructions 114. In some examples, influencer interface 502 may generate influencer instruction interface 124 (FIG. 1), for example, on a display screen of user device(s) 110, on a user interface (not shown) of influencer system 108 and/or on user interface 102. In some examples, influencer instruction interface 124 may be configured to generate a specialized GUI for the presentation, input, manipulation and/or selection of data/information in one or more windows of a display screen. In some examples, influencer instruction interface 124 may include a software application having specially programmed instructions configured to render the GUI.

In some examples, the data/information presented to influencer(s) 120 may include at least a portion of electronic patient journey plan 112, influencer instruction(s) 114, requests for updates, confirmation and/or status on actions (e.g., among action(s) 510), intervention(s) and/or interaction(s) expected to be performed by a respective influencer 120 (per influencer instruction(s) 114), one or more reminders to a respective influencer 120 (per influencer instruction(s) 114), any changes in influencer instruction(s) 114, scheduling of one or more intervention(s) and/or interaction(s) and/or any other suitable information regarding the patient's healthcare journey. In some examples, influencer interface 502 may be configured to present electronic journey plan 112 as well as one or more additional fields for collecting information from influencer(s) 120 for generating influencer instruction(s) 114 (at least in part). Any input of data/information by influencer(s) 120 may be provided, via influencer interface 502, to processor 504 for further processing and/or storage in storage 506.

Processor 504 may be configured receive electronic patient journey plan 112 (e.g., via an input/output interface) and may identify one or more influencer(s) 120, for example, based on roles and/or assignments to stakeholders identified in electronic journey plan 112. Based on the identified influencer(s) 120, processor 504 may cause influencer interface 502 to present data/information associated with the identified influencer(s) 120. For example, a portion of electronic patient journey plan 112 that may be relevant to an identified influencer 120 may be displayed. In other examples, all of the identified influencer(s) 120 may be presented with the same data/information. In some examples, additional fields for prompting input by the identified influencer(s) 120 may request input of different information depending on the role(s), assignment(s) and or action(s) 510 in electronic journey plan 112.

In some examples, processor 504 may be configured to generate one or more influencer instruction(s) 114 based on analysis and information provided by the identified influencer(s) 120 in response to the presentation of at least a portion of electronic patient journey plan 112 via influencer interface 502. Influencer instruction(s) 114 may include, without being limited to, reminder(s), intervention(s) and/or interaction(s) for specific influencer(s) 120, based on role(s) assignment(s) and/or action(s) 510 of electronic patient journey plan 112 and, in some examples, any information provided by influencer(s) 120.

Processor 504 may also be configured to trigger any reminder(s), update(s) and/or status request(s) for feedback by influencer(s) 120, for example by monitoring influencer instruction(s) 114 (e.g., stored in storage 506) over the course of electronic patient journey plan 112. In some examples, processor 504 may generate and submit influencer feedback 508, determined from responses or lack of responses (e.g., feedback) received from influencer(s) 120, to role-based optimizer 106. In some examples, role-based optimizer 106 may process influencer feedback 508, and may provide an updated electronic patient journey plan 112 to influencer system 108. For example, if one of influencer(s) 120 fails to complete an interaction noted in influencer instruction(s) 114, such a response (or lack of any response) from the particular influencer 120 may cause an update to electronic patient journey plan 112 and a corresponding change in one or more of influencer instruction(s) 114 among one or more of influencer(s) 120. In this manner, influencer system 108, together with role-based optimizer 106, may provide one or more course corrections over the patient's healthcare journey.

Processor 504 may include, without being limited to, a microprocessor, a central processing unit, an application specific integrated circuit (ASIC), a field programmable gate array (FPGA), a digital signal processor (DSP) and/or a network processor. Processor 504 may be configured to store electronic patient journey plan 112, identification information of influencer(s) 120 (e.g., an identifier, an email address, any other suitable contact information), influencer instruction(s) 114 (including any reminders), influencer feedback 508 and any other suitable information in storage 506. Storage 506 may include, without being limited to, at least one of a database, a ROM, a RAM, a flash memory, a DRAM and a SRAM.

In operation, influencer system 108 may receive electronic patient journey plan 112 and may generate influencer instruction(s) 114 (e.g., reminders, interventions and/or interactions) based on analysis by influencer(s_120 (e.g., one or more among personal influencer(s) 512 and institutional influencer(s) 514). Influencer system 108 allows stakeholders (including the patient's friends, family as well as the institutional partners) embedded within electronic patient journey plan 112 to perform relevant action(s) 510 identified (by role-based optimizer 106) for the success of the desired outcome. In general, influencer system 108 may be capable of affecting one or more changes in a patient's healthcare journey via one or more software interventions or actions, via one or more roles and institutions. Influencer system 108 may also course-correct actions based on influencer feedback 508 to role-based optimizer 106.

Referring back to FIG. 1, roles defined in system 100 may include one or more human or institutional entities. A care team, in some examples, may also include family members, friends and other closely relevant individuals, directly or indirectly related. A recommendation may be performed for one or more clinical and/or behavioral conditions and/or specialties. Factors 402 (FIG. 4) for the optimization may be based, in some examples, on data collected in the past, data collected in real-time or predicted and validated for events and interactions in a near future. Institutions may include, without being limited to hospitals, clinics, health systems, insurers, affinity groups and associations, employers and/or government bodies.

In some examples, system 100 may be configurable to function across various language-specific roles, international roles and related cultural and behavioral factors, personalization and psychosocial beliefs, attitudes and sensitivities.

In some examples, interaction between system 100 and a navigator (i.e., a user) as well as between system 100 and each role may be via text, voice, visual, gesture-based and/or any other suitable sensory communication channel.

Some portions of above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations and their associated components may be embodied in specialized software, firmware, specially-configured hardware or any combinations thereof.

It may be appreciated that the operations shown in FIGS. 1-5 may be performed by processing logic that may comprise hardware (e.g., circuitry, dedicated logic, programmable logic, microcode, etc.), software (such as instructions run on a processing device), or a combination thereof.

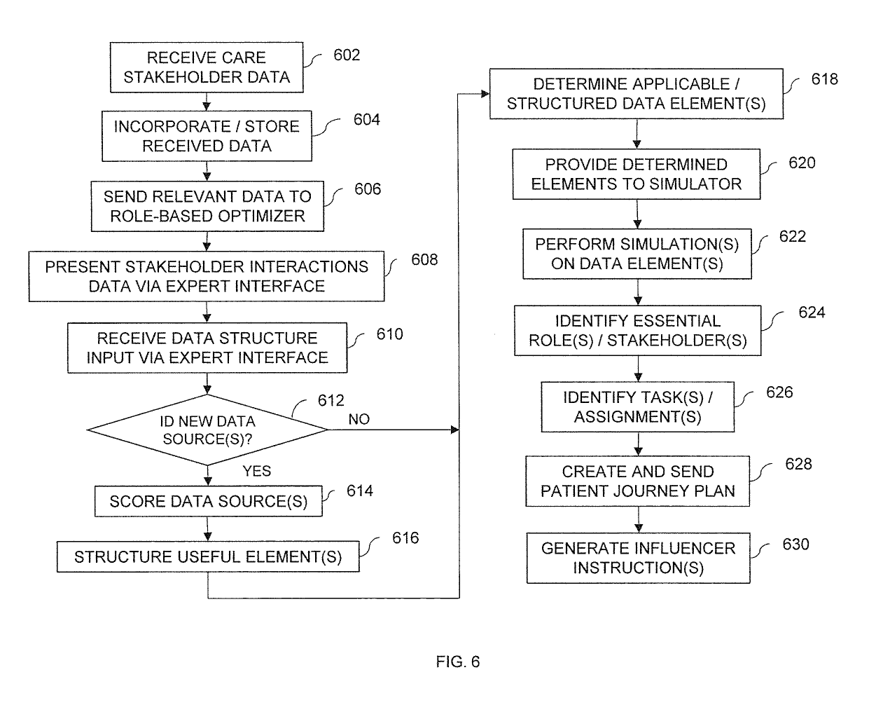

As illustrated in FIG. 6, the method shown may be performed by processing logic that may comprise hardware (e.g., circuitry, dedicated logic, programmable logic, microcode, etc.), software (such as instructions run on a processing device), or a combination thereof. In one embodiment, the method shown in FIG. 6 may be performed by one or more specialized processing components associated with components of journey optimization system 100 of FIGS. 1-5. FIG. 6 is described with respect to FIGS. 1-5.

Referring next to FIG. 6, FIG. 6 illustrates an example method for journey optimization, including for the patient and the care team via multi-faceted journey optimization system 100. At step 602, a user, via user interface 102, may enter any suitable relevant data about care stakeholders including, but not limited to, the patient, a care team, physician(s) and facility(s) regarding clinical, behavioral, psychosocial, organizational and economic determinants. At step 604, data input to user interface 102 may be appended to and enhanced by available data and metadata on all available stakeholders, via data warehouse 104. At step 606, all suitable and/or relevant information may be sent to role-based optimizer 106.

At steps 608-618, role-based optimizer 106 may identify new data sources and elements applicable to the specific optimization in order to run suitable simulations (e.g., via simulator 432). For example, at step 608, stakeholder interactions data (e.g., stored in storage 420) may be presented to expert(s) 118, for example, via expert interface(s) 422. At step 610, data structure information may be received from expert(s) 118, which may be used to develop data structure definition(s) 424.

At step 612, new data source identifier 426 may determine whether any new data sources 116 are identified. If, at step 612, no new data sources 116 are identified, step 612 may proceed to step 618.

If, at step 612, new data source identifier 426 identifies at least one new data source 116, step 612 may proceed to step 614. At step 614, data source scorer 428 may score the identified data source(s) 116. At step 616, element structurer 430 may structure any useful data elements. Step 616 may proceed to step 618.

At step 618, mapper 414 may determine any applicable and structured data element(s) (e.g., stored in storage 420).

At step 620, the structured and applicable data elements may be provided as input to simulator 432 for performing at least one simulation process. At step 622, one or more simulations may be performed, for example, by simulator 432, on the applicable data elements. At step 624, based on the simulations, role identifier 434 may identify the essential (desired) roles and/or desired stakeholders. At step 626, role assigner 436 may identify assignments and/or tasks for each of the roles and/or stakeholders. At step 628, role-based optimizer 106 may combine and package the identified roles, assignments and tasks to create electronic patient journey plan 112.

At step 628, role-based optimizer 106 may also send electronic patient journey plan 112 to influencer system 108. Influencer system 108 may provide electronic patient journey plan 112 to influencer(s) 120 (for example, including to personal influencer(s) 512 and institutional influencer(s) 514), via influencer interface 502. Influencer(s) 120 (e.g., 512, 514) may, for example, be based inside of a care facility such as office staff or nurses (e.g., institutional influencer(s) 514), or may be personal care coaches to the patient such as friends or family (e.g., personal influencer(s) 512).

At step 630, influencer system 108 may generate influencer instruction(s) 114 (e.g., reminders, interventions, interactions) for influencer(s) 512 and/or influencer(s) 514 to perform. This availability or access is visible through influencer system 108, which allows personal influencer(s) 512 and institutional influencer(s) 514 to interact with electronic patient journey plan 112 in an effort to maintain the progress of patient journey plan 112 and subsequent outcomes.

Systems and methods of the present disclosure may include and/or may be implemented by one or more specialized computers including specialized hardware and/or software components. For purposes of this disclosure, a specialized computer may be a programmable machine capable of performing arithmetic and/or logical operations and specially programmed to perform the functions described herein. In some embodiments, computers may comprise processors, memories, data storage devices, and/or other commonly known or novel components. These components may be connected physically or through network or wireless links. Computers may also comprise software which may direct the operations of the aforementioned components. Computers may be referred to with terms that are commonly used by those of ordinary skill in the relevant arts, such as servers, personal computers (PCs), mobile devices, and other terms. It will be understood by those of ordinary skill that those terms used herein are interchangeable, and any special purpose computer capable of performing the described functions may be used.

Computers may be linked to one another via one or more networks. A network may be any plurality of completely or partially interconnected computers wherein some or all of the computers are able to communicate with one another. It will be understood by those of ordinary skill that connections between computers may be wired in some cases (e.g., via wired TCP connection or other wired connection) or may be wireless (e.g., via a WiFi network connection). Any connection through which at least two computers may exchange data can be the basis of a network. Furthermore, separate networks may be able to be interconnected such that one or more computers within one network may communicate with one or more computers in another network. In such a case, the plurality of separate networks may optionally be considered to be a single network.

The term “computer” shall refer to any electronic device or devices, including those having capabilities to be utilized in connection with journey optimization system 100 (including components 102-110 and/or 116), such as any device capable of receiving, transmitting, processing and/or using data and information. The computer may comprise a server, a processor, a microprocessor, a personal computer, such as a laptop, palm PC, desktop or workstation, a network server, a mainframe, an electronic wired or wireless device, such as for example, a telephone, a cellular telephone, a personal digital assistant, a smartphone, an interactive television, such as for example, a television adapted to be connected to the Internet or an electronic device adapted for use with a television, an electronic pager or any other computing and/or communication device.

The term “network” shall refer to any type of network or networks, including those capable of being utilized in connection with journey optimization system 100 described herein, such as, for example, any public and/or private networks, including, for instance, the Internet, an intranet, or an extranet, any wired or wireless networks or combinations thereof.

The term “computer-readable storage medium” should be taken to include a single medium or multiple media that store one or more sets of instructions. The term “computer-readable storage medium” shall also be taken to include any medium that is capable of storing or encoding a set of instructions for execution by the machine and that causes the machine to perform any one or more of the methodologies of the present disclosure.

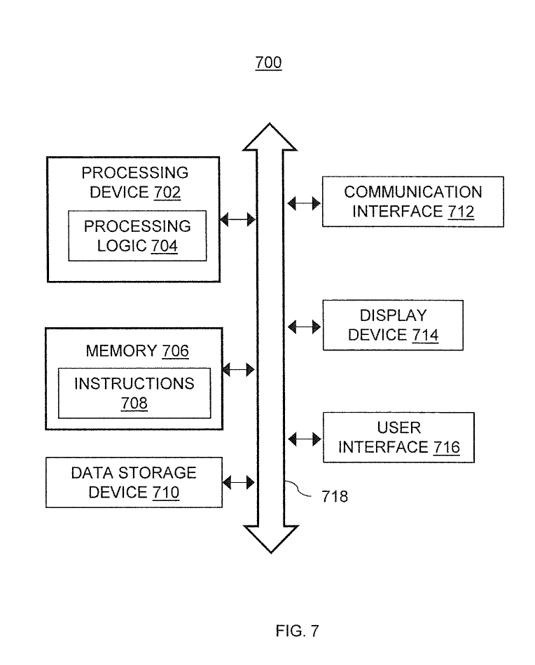

FIG. 7 illustrates a functional block diagram of a machine in the example form of computer system 700 within which a set of instructions for causing the machine to perform any one or more of the methodologies, processes or functions discussed herein may be executed. In some examples, the machine may be connected (e.g., networked) to other machines as described above. The machine may operate in the capacity of a server or a client machine in a client-server network environment, or as a peer machine in a peer-to-peer (or distributed) network environment. The machine may be any special-purpose machine capable of executing a set of instructions (sequential or otherwise) that specify actions to be taken by that machine for performing the functions describe herein. Further, while only a single machine is illustrated, the term “machine” shall also be taken to include any collection of machines that individually or jointly execute a set (or multiple sets) of instructions to perform any one or more of the methodologies discussed herein. In some examples, one or more components of journey optimization system 100 (user interface 102, data warehouse 104, role-based optimizer 106, influencer system 108, user device(s) 110 and/or data source(s) 116) may be implemented by the example machine shown in FIG. 7 (or a combination of two or more of such machines).

Example computer system 700 may include processing device 702, memory 706, data storage device 710 and communication interface 712, which may communicate with each other via data and control bus 718. In some examples, computer system 700 may also include display device 714 and/or user interface 716.

Processing device 702 may include, without being limited to, a microprocessor, a central processing unit, an ASIC, a FPGA, a DSP and/or a network processor. Processing device 702 may be configured to execute processing logic 704 for performing the operations described herein. In general, processing device 702 may include any suitable special-purpose processing device specially programmed with processing logic 704 to perform the operations described herein.

Memory 706 may include, for example, without being limited to, at least one of a read-only memory (ROM), a RAM, a flash memory, a DRAM and a SRAM, storing computer-readable instructions 708 executable by processing device 702. In general, memory 706 may include any suitable non-transitory computer readable storage medium storing computer-readable instructions 708 executable by processing device 702 for performing the operations described herein. For example, computer-readable instructions 708 may include operations performed by components 102-110 of journey optimization system 100), including operations shown in FIG. 6). Although one memory device 706 is illustrated in FIG. 7, in some examples, computer system 700 may include two or more memory devices (e.g., dynamic memory and static memory).

Computer system 700 may include communication interface device 712, for direct communication with other computers (including wired and/or wireless communication) and/or for communication with a network. In some examples, computer system 700 may include display device 714 (e.g., a liquid crystal display (LCD), a touch sensitive display, etc.). In some examples, computer system 700 may include user interface 716 (e.g., an alphanumeric input device, a cursor control device, etc.).

In some examples, computer system 700 may include data storage device 710 storing instructions (e.g., software) for performing any one or more of the functions described herein. Data storage device 710 may include any suitable non-transitory computer-readable storage medium, including, without being limited to, solid-state memories, optical media and magnetic media.

While the present disclosure has been discussed in terms of certain embodiments, it should be appreciated that the present disclosure is not so limited. The embodiments are explained herein by way of example, and there are numerous modifications, variations and other embodiments that may be employed that would still be within the scope of the present disclosure.

1. An optimization system comprising:
a user interface configured to receive data comprising multi-factor determinants of a patient and a plurality of treatment personnel, the multi-factor determinants including at least one of clinical, behavioral, psychosocial, organizational and economic characteristics;
an optimizer configured to identify one or more personnel among the plurality of treatment personnel to form a care team, one or more roles for each selected personnel of the care team and one or more actions for each of the care team and the patient, to generate an electronic patient journey plan for the patient, based on optimization of the multi-factor determinants of the patient and the plurality of treatment personnel according to one or more optimization algorithms; and
an influencer system configured to determine one or more software influencer instructions to be performed by one or more designated influencers based on the electronic patient journey plan.

2. The optimization system of claim 1, wherein the one or more software influencer instructions include at least one of one or more interventions, one or more interactions and one or more reminders associated with each of the one or more designated influencers.

3. The optimization system of claim 1, wherein the influencer system comprises an influencer interface and a processor, the processor configured to cause the influencer interface to display at least one of the electronic patient journey plan, the one or more software influencer instructions and at least one update to the one or more software influencer instructions.

4. The optimization system of claim 3, wherein the influencer system is configured to receive input from at least one of the one or more designated influencers via the influencer interface, and the processer is configured to generate at least one corresponding instruction among the one or more software influencer instructions responsive to the received input.

5. The optimization system of claim 3, wherein the processor of the influencer system is configured to present, via the influencer interface, to at least one among the one or more designated influencers, at least one of a reminder and a request for information associated with the one or more influencer instructions.

6. The optimization system of claim 5, wherein the processor of the influencer system is configured to monitor information including at least one of the one or more software influencer instructions, any response and any lack of response from among the one or more designated influencers in response to the at least one of the reminder and the request for information.

7. The optimization system of claim 6, wherein the processor of the influencer system is configured to generate influencer feedback information based on the monitored information and send the feedback information to the optimizer, and the optimizer is configured to generate an updated electronic patient journey plan in response to the feedback information received from the influencer system.

8. The optimization system of claim 1, wherein the electronic patient journey plan indicates one or more steps for navigation of a healthcare process associated with the patient.

9. The optimization system of claim 1, wherein the optimizer is configured to optimize the multi-factor determinants according to at least one predefined outcome.

10. The optimization system of claim 1, wherein the multi-factor determinants further include at least one of patient disease stage information, patient diagnosis information and patient adherence information.

11. The optimization system of claim 1, wherein the one or more optimization algorithms include at least one of machine learning, artificial intelligence and statistical processing techniques.

12. The optimization system of claim 1, wherein the optimizer is configured to obtain data associated with interactions between one or more patients and one or more healthcare personnel from among one or more data sources, and the optimization is based at least in part on the obtained data.

13. The optimization system of claim 12, wherein the optimizer further includes an expert interface configured to receive one or more definitions associated with one or more of data elements and at least one source among the one or more data sources, and the optimizer is configured to generate at least one data structure definition based on the received one or more definitions.

14. The optimization system of claim 12, wherein the optimizer is configured to identify at least one additional data source based on at least one of web discovery and web scraping.

15. The optimization system of claim 14, wherein the optimizer is configure to score the at least one identified additional data source and to structure at least one data element of the at least one identified additional data source.

16. A method for creating an optimized patient healthcare journey plan, the method comprising:
receiving, via a user interface of an optimization system, data comprising multi-factor determinants of a patient and a plurality of treatment personnel, the multi-factor determinants including at least one of clinical, behavioral, psychosocial, organizational and economic characteristics;
generating, by an optimizer of the optimization system, an electronic journey plan for the patient, by identifying one or more personnel among the plurality of treatment personnel to form a care team, one or more roles for each selected personnel of the care team and one or more actions for each of the care team and the patient, based on optimization of the multi-factor determinants of the patient and the plurality of treatment personnel according to one or more optimization algorithms; and
determining, by an influencer system of the optimization system, one or more software influencer instructions to be performed by one or more designated influencers based on the electronic patient journey plan.

17. The method of claim 16, wherein the one or more software influencer instructions include at least one of one or more interventions, one or more interactions and one or more reminders associated with each of the one or more designated influencers.

18. The method of claim 16, the method further comprising:
displaying, via an influencer interface of the influencer system, information including at least one of the electronic patient journey plan, the one or more software influencer instructions and at least one update to the one or more software influencer instructions.

19. The method of claim 18, the method further comprising:
receiving input from at least one of the one or more designated influencers via the influencer interface responsive to the displayed information; and
generating, by the influencer system, at least one corresponding instruction among the one or more software influencer instructions responsive to the received input.

20. The method of claim 18, the method further comprising:
presenting, via the influencer interface, to at least one among the one or more designated influencers, at least one of a reminder and a request for information associated with the one or more influencer instructions.

21. The method of claim 20, the method further comprising:
monitoring, by the influencer system, information including at least one of the one or more software influencer instructions, any response and any lack of response from among the one or more designated influencers in response to the at least one of the reminder and the request for information.

22. The method of claim 22, the method further comprising:
generating, by the influencer system, influencer feedback information based on the monitored information;
sending the feedback information to the optimizer; and
generating, by the optimizer, an updated electronic patient journey plan in response to the feedback information received from the influencer system

23. The method of claim 16, wherein the optimization of the multi-factor determinants includes optimizing the multi-factor determinants according to at least one predefined outcome.

24. The method of claim 16, wherein the multi-factor determinants further include at least one of patient disease stage information, patient diagnosis information and patient adherence information.

25. The method of claim 16, wherein the one or more optimization algorithms include at least one of machine learning, artificial intelligence and statistical processing techniques.

26. The method of claim 16, the method further comprising:
obtaining, by the optimizer, data associated with interactions between one or more patients and one or more healthcare personnel from among one or more data sources, wherein the optimization is based at least in part on the obtained data.

27. The method of claim 26, the method further comprising:
receiving, via an expert interface of the optimizer, one or more definitions associated with one or more of data elements and at least one source among the one or more data sources; and
generating, by the optimizer, at least one data structure definition based on the received one or more definitions.

28. The method of claim 26, the method further comprising:
identifying at least one additional data source based on at least one of web discovery and web scraping.

29. The method of claim 28, the method further comprising:
scoring the at least one identified additional data source; and
structuring at least one data element of the at least one identified additional data source.

30. A non-transitory computer readable medium storing computer readable instructions that, when executed by one or more processing devices, cause the one or more processing devices to perform the functions comprising:
receiving, via a user interface, data comprising multi-factor determinants of a patient and a plurality of treatment personnel, the multi-factor determinants including at least one of clinical, behavioral, psychosocial, organizational and economic characteristics;
generating an electronic patient journey plan for the patient, by identifying one or more personnel among the plurality of treatment personnel to form a care team, one or more roles for each selected personnel of the care team and one or more actions for each of the care team and the patient, based on optimization of the multi-factor determinants of the patient and the plurality of treatment personnel according to one or more optimization algorithms; and
determining one or more software influencer instructions to be performed by one or more designated influencers based on the electronic patient journey plan.
